(19) United States
(12) Patent Application Publication
(10) Pub. No.: US 2008/0207537 A1
(43) Pub. Date: Aug. 28, 2008

(54) Topical Ungual Formulations

(75) Inventors: Robert Turner, Portsmouth (GB); Marc Barry Brown, Watford (GB); Stuart Allen Jones, Blackheath (GB)

Correspondence Address: GREENLEE WINNER AND SULLIVAN P C, 4875 PEARL EAST CIRCLE, SUITE 200, BOULDER, CO, 80301, US

(73) Assignee: MedPharm Limited, Oxfordshire (GB)

(21) Appl. No.: 11/916,514

(22) PCT Filed: Jun. 6, 2006
(86) PCT No.: PCT/GB2006/002064

§ 371 Date: May 2, 2008

(30) Foreign Application Priority Data
Jun. 6, 2005 (GB) 0511499.6

(51) Int. Cl. A61K 31/7048; A61K 47/22; A61K 47/20; A61K 47/18; A61K 47/14; A61K 47/12; A61K 47/02; A61K 31/343; A61K 31/513; A61P 31/10; A61K 31/436; A61K 31/519; A61K 31/506; A61K 31/5375; A61K 31/4174; A61K 31/496; A61K 31/4196; A61K 31/4164; A61K 31/135

(52) U.S. Cl. 514/31; 514/785; 514/784; 514/772; 514/788; 514/769; 514/239.5; 514/399; 514/254.07; 514/383; 514/655; 514/649; 514/462; 514/256; 514/274; 514/249; 514/290

(57) ABSTRACT

Application of a reducing agent followed by an oxidising agent to a nail substantially increases the permeability thereof, thereby enabling the passage of drugs across the nail.

The present invention relates to topical formulations for ungual application, the formulations comprising a drug and a penetration enhancer.

Onychomycosis is the generic term for fungal infections of the nail plate or nail bed, and is responsible for up to 50% of nail disorders. Both the fingernails and the toenails can be affected. In Europe, the condition is currently thought to affect approximately 5% of the population and is becoming ever more prevalent. This increase is mainly attributed to an aging population, since onychomycosis is much more common amongst the elderly. Other contributory factors include poor footwear and increased use of communal leisure facilities.

The pathogens most often responsible for causing onychomycosis are dermatophytes, which are thought to be responsible for more than 90% of all cases. Trichophyton rubrum (toe nails 56%—finger nails 36%) and Trichophyton mentagrophytes (toe nails 19%—11% finger nails) are especially common. Yeast infections are far less common, but are usually associated with Candida albicans (toe nails 10%—30% finger nails).

A second, relatively common nail disorder is psoriasis. Psoriasis is most familiar as an inflammatory disease of the skin, but most patients who suffer from skin psoriasis also suffer from nail psoriasis. It is rare for patients to only suffer from nail psoriasis. Psoriasis is most common in Europe and North America, where it affects around 3% of the population.

Although nail disorders are rarely life threatening, they can be very painful and disfiguring for the sufferer. Common symptoms include changes to the nail colour, often to a yellow/green or darker colour, and the collection of debris under the nail, causing a foul smell. Additionally, the nail may thicken and become flaky. Such aesthetic indicia alone can have a serious effect on the quality of life of the sufferer. In addition to these symptoms, the condition can be very painful. Thick toenails, in particular, may cause discomfort in shoes and may even make standing and walking uncomfortable for some sufferers.

Effective treatment of onychomycosis, and other nail disorders, is seriously hampered by the fact that the site of infection is effectively shielded by the nail plate, which consists mainly of keratins, a fibrous group of proteins. The keratin fibres are held together by globular proteins rich in cysteine, whose disulphide bonds act in a glue-like manner, and are responsible for much of the nail plate's integrity. Any effective treatment must be able to overcome the obstacle presented by the hard and rigid nail plate and deliver an active species to the nail bed.

Known methods of treatment fall into three general categories. The first involves the removal of all or part of the affected nail to expose the site of infection. Removal may be surgical or chemical. Chemical removal might involve the application of urea to the nail plate within an occlusive dressing over a short period. Urea acts to unfold the proteins within the nail. The nail becomes soft and detaches from the nail bed. However, the traumatic and painful nature of such treatment means that it is unpopular, and is only used as a last resort.

The second general method of treatment is the oral administration of an appropriate drug. There are currently four main oral therapies available for the treatment of onychomycosis. These are Griseofulvin (Grisovine®, GSK), Ketoconazole (Nizoral®, Janssen-Cilag), Itraconazole (Sporanox®, Janssen-Cilag) and Terbinafine (Lamisile®, Novartis). For psoriasis, oral agents such as methotrexate, etretinate and ciclosporin can be effective.

Griseofulvin has been available since the 1950's. Due to its fungistatic activity against dermatophytes, long treatment periods are required (9-12 months for toenail infections). Cure rates are low, and relapse rates are high. Ketoconazole was the first imidazole-based drug to be introduced for the treatment of onychomycosis in the 1980's. However, due to its hepatotoxicity, its use is now restricted to fingernail infections that have failed to respond to other therapies. The more recent antifungal agents, Itraconazole and Terbinafine, are more effective in the oral treatment of onychomycosis, with higher mycological cure rates and shorter treatment periods than previously observed.

The third method involves the topical application of a composition to the nail plate. Topical therapies for onychomycosis, currently on the market, include Amorolfine (Loceryl®, Galderma) and Ciclopirox (Penlac®, Dermik). As used herein, the terms ‘topical’ and ‘topically’ indicate application to a surface, such as skin or nail, in contrast to systemic application, which is normally by ingestion or injection.

Topical treatments for nail disorders are most preferable, in principle, as they do not carry the same risks as systemic drugs, such as hepatotoxicity, and are less painful and disfiguring than treatments involving full or partial nail removal. However, to be effective, the active species must be able to penetrate the nail plate in sufficient quantities, such that efficacious concentrations of the active species can reach the deeper layers of the nail plate, as well as the nail bed itself. Probably as a result of poor drug penetration, the topical treatments currently available are relatively ineffective, and are associated with long treatment times and low cure rates.

In comparison with the thin stratum corneum of the skin, the nail plate is much thicker. This means that there is a much longer diffusion pathway for drug delivery to the nail bed. In addition, nail does not act like a lipoidal barrier, but more like a concentrated hydrogel. The disulphide bonds of the cysteine-rich proteins are largely responsible for the integrity and structure of the nail and for its barrier properties. The development of effective topical treatments for nail disorders, therefore, represents a much greater challenge than the development of topical skin treatments.

There are several factors that influence the rate of diffusion of drugs into and through the nail plate. These factors include the size of the diffusing species, the hydrophilicity/lipophilicity of the diffusing species, and the nature of the vehicle. Additionally, penetration may be enhanced by effectively reducing the barrier that drugs must diffuse across in order to reach the site of infection at the nail bed. The barrier may be reduced physically or chemically.

Physical reduction of the barrier involves partial or full removal of the nail, or filing away the upper layer of the nail plate. Such procedures are undesirable, since they are both painful and disfiguring.

A more acceptable approach involves the use of chemical enhancers which, when applied to the nail, interact with and modify the nail structure such as to reduce the barrier to drug permeation and increase the rate of diffusion of the active species into and through the nail plate. Urea is commonly used as a nail penetration enhancer, given that it has the ability to chemically remove nail plates. Other approaches have focussed on the disulphide bonds in the nail, and disrupters of these bonds include acetylcysteine and mercaptoethanol.

At present, there are no topical treatments available that give satisfactory results in the treatment of conditions affecting the lower layers of the nail plate and the nail bed.

U.S. Pat. No. 6,664,292 discloses topical compositions for the treatment of pathological conditions of the nail. The compositions consist of a lower alcohol and a lower carboxylic acid.

U.S. Pat. No. 5,753,256 discloses a plaster for the treatment of nail mycosis. The plaster includes an active compound and a permeation enhancer. Permeation enhancers disclosed are sulphoxides, lactic acid, salicylic acid, propylene glycol, dimethylformamide, dimethylacetamide and sodium dodecylsulphate.

US Application No. 2003/0235541 discloses an aqueous, basic formulation for topical treatment of onychomycosis.

US Patent Application No. 2001/0049386 discloses a method of treating onychomycosis wherein a tissue softening composition comprising urea and an antifungal composition are administered to an infected area around a nail, either in one or in separate compositions, concurrently or non-concurrently.

WO 99/40955 discloses a pressure sensitive adhesive matrix patch for the treatment of onychomycosis. Skin permeation enhancers are optionally included in the patch.

GB-A-2278056 discloses higher esters and amides of thioglycolic acid as penetration enhancers for dermal use, and discloses formulations suitable for the treatment of onychomycosis. However, these formulations can only be applied peri-ungually. In addition, these treatments still require the systemic presence of drug, as the formulations are for transdermal administration so that, although the treatment can be applied locally, the drug will still enter the bloodstream.

WO 99/49895 discloses that thioglycolic acid is capable of reducing keratin in the nail and, therefore, can be used to improve the diffusion of drugs through the nail to the nail bed.

DE 1000567 discloses the use of thioglycolic acid in combination with sodium iodide to reduce ungual keratin, while EP 0712633A1 simply discloses the use of thioglycolic acid as a skin permeation enhancer.

US 2003/0007939 discloses a pharmaceutical composition of hydrogen peroxide and at least one other dermatological agent to enhance the penetration in the skin, scalp, hair and nail. The use of a reducing agent is not described.

EP 0,425,507 discloses a composition for treating abnormal or damaged conditions of the epithelium, including skin, which comprise an activated protein, an oxidising agent, including hydrogen peroxide, and a reducing agent, including thioglycolic acid.

Thus, there is still a need for effective, topically applied formulations for the treatment of nail conditions.

We have now, surprisingly, discovered that a reducing agent, such as thioglycolic acid (TA) and an oxidising agent, such as hydrogen peroxide, can be applied to the nail one after another to enhance drug penetration. In ordinary circumstances, the combination of the two agents generally results in an extreme chemical reaction. However, on the nail, it appears to be safe to combine these agents.

Thus, in a first aspect, the present invention provides the use of a, preferably liquid, preparation of each of a reducing agent and an oxidising agent in the manufacture of a medicament for the treatment of an ungual condition treatable by a drug, said preparations being separately disposed and for sequential administration to the nail of a patient in the order of reducing agent followed by oxidising agent, the said drug being disposed in one or other preparation or, optionally, in a third preparation, where the drug is additional to the reducing or oxidising agent.

The, or a, reducing agent and/or the, or an, oxidising agent may be selected from known drugs, such as anti-fungals and anti-psoriatics, that have appropriate reducing or oxidising properties, but it is generally preferred that an extra drug be employed, as discussed below.

It is preferred that the reducing and oxidising agents be sufficiently powerful that a simple combination of the two preparations would lead to an extreme reaction, such as a substantially exothermic, or even explosive, reaction. When applied to the nail in sequential fashion, reducing agent followed by oxidising, it has now, surprisingly, been established that such combinations can be safely administered without immediate visible indicia of an extreme reaction. It will be appreciated that certain reducing and oxidising agents will be too powerful to apply safely to a nail, or will result in undesirable reactions, such as discoloration, which may be unacceptable for some patients. However, provided that each agent is individually acceptable to apply to a nail, then combinations are also acceptable, even where a combination of the two, in the absence of nail would result in an extreme reaction. The agents thioglycolic acid and hydrogen peroxide provide an example of one such combination. Other agents/combinations may be readily selected by those skilled in the art, by applying the above criteria.

Suitable concentrations for either agent are selected independently, and are generally between 1 and 50% w/v, with a preferred range being between 5 and 30%. More preferably the concentration of each is separately selected from a range between 10 and 25%, and particularly 5 and 15%.

In a preferred embodiment, the desired strength of the reducing and oxidising agents can be selected by reference to their reduction potentials. As is well known in the art, the reduction potential of a particular species indicates the ability of that species to accept electrons, thereby providing an indication of its ability to be reduced.

The reduction potential of the oxidising and reducing agents according to the present invention can be measured by any procedure known to those skilled in the art. A preferred method is to measure the reduction potential of a solution of a particular agent, in water or in a mixture of water and ethanol, relative to a silver/silver chloride electrode, at r.t.p. and calibrated against redox standards.

According to a preferred embodiment of the invention, the reduction potential of the oxidising agent, when measured in accordance with the above protocol, is preferably less than −50 mV, more preferably less than −100 mV, and most preferably less than −200 mV. The reduction potential of the reducing agent is preferably greater than +20 mV, more preferably greater than +50 mV and most preferably greater than +75 mV.

As demonstrated in the accompanying Examples, the reduction potential of solutions containing either agent may be adjusted by altering the concentration of the oxidising or reducing agent present in the solution, or by adjusting the pH of the solution, if desired.

Thus, in a preferred embodiment, the reducing agent is prepared as an alkaline preparation, with a pH of between 7 and 14. More preferably, the pH is between 8 and 13, with a pH of between 9 and 12 being particularly preferred, as this generally maximises the reduction potential of a reducing compound.

Conversely, while the pH of the oxidising agent is generally of less importance, it is preferred that the pH be between 1 and 7, with a pH of between 2 and 5 being more preferred, as this tends to maximise the oxidation potential (−ve reduction potential). In any event, it is preferred that neither preparation be so acidic/caustic that inadvertent splashing of the skin leads to pharmaceutically and/or cosmetically unacceptable damage.

However, it will be appreciated that the pH of either formulation may be modified in order to achieve a desired effect, such as to moderate reduction potential or rate of reaction, so that the above-indicated preferences apply generally when there are no other relevant considerations regarding pH.

In general, formulations of the reducing and oxidising compounds are so selected that reduction potentials are within the ranges indicated above, and generally so as to maximise the +ve or −ve potential, as appropriate. While it is generally preferred that the reduction potential be optimised by concentration, it will be appreciated that formulations having low concentrations may be provided where evaporation of the solvent and/or co-solvent will lead to higher concentrations in situ.

Preferred concentrations of agents are readily ascertained in accordance with the techniques exemplified in the accompanying Examples. For example, a preferred concentration range of thioglycolic acid is at least 0.1% and up to 20% and higher; urea-H₂O₂ at least 5% and up to 40% and above, H₂O₂ at least 20% and up to 100%, and DTT at least 0.05% and up to 20% and above. Preparations formulated with ethanol, or other volatile solvent or co-solvent, may be prepared with lower concentrations of the reducing or oxidising agent, as the concentration of the agent will increase on application to the nail when the preparation is applied thereto.

It is particularly surprising that the application of the two components appears to be synergistic in that there is no reason to suspect that the enhanced permeability generated by either component is sufficiently different to be able to be additive with the other. Indeed, this mode of action is not implicit in the art, as the application of the oxidising agent first generally leads to no appreciable enhancement of nail permeability above that of the more effective of the two components. However, use of the reducing agent first appears to lead to such synergy that the effect is often greater than the combined effect of both agents. This effect may be, but is not always, reflected in a measure of increased liquid uptake by the nail (infra).

Preferred reducing agents include ammonium thioglycolate, calcium thioglycolate, sodium thioglycolate, thioglycolic acid (TA), and dithiothreitol (DTT), ascorbic acid, hydroquinone, mercaptoethanol, glutathione, L-cysteine, taurine, aminomethanesulphonic acid, cysteic acid, cysteinesulphinic acid, ethanedisulphonic acid, ethanesulphonic acid, homotaurine, hypotaurine, isethionic acid, mercaptoethanesulphonic acid, N-inethyltaurine (MTAU), as well as simple derivatives thereof.

By “simple derivative” is meant a salt, ester or amide of the compound. Simple derivatives of thioglycolic acid are especially preferred. Any further alkyl component is preferably a lower alkyl having 1 to 6 carbon atoms in total, but more preferably having 1 to 4 carbon atoms and, most preferably, 1, 2 or 3 carbons.

The reducing agent is preferably thioglycolic acid or a derivative thereof and, as such, the reducing agent is commonly referred to herein as such, although it will be understood that any such reference will also include other reducing agents, unless otherwise clear or apparent.

The oxidising agent may be any that is suitable, including urea, hydrogen peroxide, potassium persulphate, thiouracil, p-coumeric acid, glycolic acid, oxalic acid, cineol, peroxydone, chlorine dioxide, ammonium dichromate, ammonium nitrate, ammonium perchlorate, ammonium permanganate, barium bromate, barium chlorate, barium peroxide, cadmium chlorate, calcium chlorate, calcium chromate, calcium perchlorate, chromium nitrate, cobalt nitrate, silver oxide, periodic acid, and pyridine dichromate. Hydrogen peroxide is preferred, and an addition compound of hydrogen peroxide and urea is more preferred.

The preparations may be administered one immediately after the other, but it is preferred to apply the preparations in the order of reducing preparation followed by oxidising preparation, and to allow the first preparation to react with the nail for a short time, before applying the oxidising preparation. A suitable time is between 10 seconds and 10 minutes, more preferably between 1 minute and 5 minutes. While such short times are acceptable, it has been found that substantial periods may be allowed to elapse between applications, and a preferred period between application of reducing and oxidising agents is between 15 and 30 hours, and more preferably 20 to 26 hours, with similar periods between subsequent applications. When such longer periods are employed, then it is preferable for the drug to be present in one or both preparations, or administered in a separate formulation but together with one or both preparations.

The drug may be present in one or both of the preparations, or may be prepared separately for administration before, during, or after application of the oxidising and reducing agents. In one embodiment, the reducing agent is applied to the nail, followed by the drug and then the oxidising agent, such that the drug is in place in the event that interaction between the oxidising and reducing agents results in permeation enhancement. It is generally preferred that the drug be present in a separate preparation, especially where prolonged exposure to either or both of oxidising and reducing agents is undesirable.

The formulations of the present invention are for ungual application, and may be formulated in any manner suitable for application to an unguis or nail.

The preparations are preferably aqueous, optionally with a co-solvent, such as ethanol or acetone. Although the co-solvent may not be necessary for the oxidising or reducing agents, it may be necessary for solubilisation of the drug.

In general, ungual formulations of the present invention may be provided as creams, ointments, gels, solutions, lotions, foams, mousses, sprays, pastes or lacquers, where they are intended for direct application to the nail. However, formulations of the invention may also be provided as solutions or powders or premixes, for example. A solution, for instance, may be applied to a dressing, such as a plaster, and then associated with the nail, in order to more accurately target the site. Alternatively, a dressing may be applied to the nail, and then the solution applied to the dressing. In this manner, for example, it may be possible to restrict the dressing entirely to the nail surface, or the dressing may be constructed in such a manner that contact between the solution and non-nail surfaces, or the site to be treated, is inhibited or prevented, either by the construction of the dressing, or by introduction of barrier means. Suitable barrier means may include certain adhesives or resins, or other suitable treatments. Where such dressings are used, then it is preferred to use separate dressings for each of the reducing and oxidising agents in order to avoid a potentially explosive reaction occurring.

Where a dressing is provided, it may also be desirable to apply a further occlusion dressing to the dressing, once the solution or other preparation of drug has been applied to the absorptive dressing, in order to prevent evaporation of the carrier, where this is undesirable.

A patch, similar in construction to a transdermal patch, but preferably a reservoir patch, may be used to provide one agent, after application of the other by way of a paint or lacquer, for example. Likewise, a powder may applied by dusting onto a lacquered or painted surface, for example.

Other powders and premixes may be further made up, as desired, into solutions or preparations for application to the nail, for example.

It will be understood that references to “nail” herein include references to any appropriate ungual surface. In humans, this will effectively only comprise fingernails and toenails, but any ungual surface is envisaged.

Suitable carriers for the agents generally include water and lower alcohols including monohydric and polyhydric alcohols. Other solvents may also be employed, such as acetone and, in general, it will be readily apparent to those skilled in the art as to which solvents are appropriate for application to the nail.

In addition to the carrier, other vehicles may be used as bulking agents, for example. While water may be employed to form the bulk of the formulation, it has been found that propylene glycol is generally associated with higher levels of ungual penetration, and is generally a more preferred bulking agent.

The preparations may be simple aqueous preparations, or may contain further ingredients, such as thickeners, stabilisation enhancers, pH modifying agents, odour inhibitors, and colourants, for example. The use of odour inhibitors may be particularly preferred where the medicament is intended for the treatment of a malodorous nail condition, which is common in such conditions, or where it is desirable to mask the odour of the medicament itself. Similarly, colourants may be included where the condition being treated results in an undesirable discolouration of the nail. In one embodiment, the preparations are prepared as varnishes that dry in situ.

In another embodiment, the reducing and/or oxidising agents, and especially thioglycolic acid, may be conjugated with the drug to be administered.

By the term “drug” is meant any active ingredient of the formulation of the invention which is able to exert a therapeutic effect on application to a nail. The therapeutic effect may only be noticeable when in association with a penetration enhancer of the invention.

Suitable drugs for use in the formulations of the invention may be for any condition associated with the unguis of the patient, but will often fall into the category of either fungal infection or a condition associated with psoriasis. The drug may be in any suitable form, and may be a solid, a liquid, or a gas, present in a preparation to be administered to the nail. Suitable gases include NO, for example.

Suitable drugs for use in the formulations of the present invention include the antifungal drugs: amorolfine, miconazole, ketoconazole, itraconazole, fluconazole, econazole, ciclopirox, oxiconazole, clotrimazole, terbinafine, naftifine, amphotericin, griseofulvin, voriconazole, flucytosine, nystatin and pharmaceutically acceptable salts and esters thereof. Particularly preferred is terbinafine.

Suitable drugs for use in connection with psoriasis include corticosteroids, 5-fluorouracil, methotrexate, etretinate, cyclosporin, tacrolimus, and derivatives thereof.

The amount of drug used is not critical to the invention, and all that is required is that the drug be able to be administered in an effective amount for the treatment or prophylaxis of an ungual condition. The amount of drug may depend on the age, sex and/or weight of the patient, but will generally be provided as a stock preparation for applying to the nail, and concentration of the drug will generally be dependent on the condition to be treated, as the main determining factor.

The amount of drug may be sufficient to eliminate the condition after one treatment, but it is generally the case that the treatment will be continued for a number of applications, so that the amount of drug may be tailored for gradual treatment according to the intended number of cycles.

In cyclical treatment, the infected nail is treated cyclically with reducing agent and oxidising agent. A preferred strategy is to treat with reducing agent on day 1, then oxidising agent on day 2, with drug administered preferably together with, or shortly after the oxidising agent, and then to repeat the process on day 3, starting with application of the reducing agent once again. Optionally, a gap may be provided between treatments, so that the repeat treatment starts again on day 4, day 5, day 6, or day 7, for example. The gap may be longer if preferred, but is preferably no more than a month, and preferably no more than 2 weeks.

It will be appreciated that the cycle may comprise two or more applications of any of the preparations. For example, the reducing agent may be applied each day for two or three consecutive days, for example, or on several occasions during one day, with the oxidising agent and drug being applied on the day following the last treatment with reducing agent, or a day after the first treatment, which may be shortly after the last treatment with reducing agent. The oxidising agent may be applied on successive days, or on several occasions during one day, and it is generally preferred that the drug be applied with, or shortly after, each treatment with oxidising agent.

Suitable concentrations of drug in preparations of the invention will be dependent on such factors as the condition to be treated, and the drug to be used and, in any case, will be readily apparent to those skilled in the art. For guidance, suitable concentrations may be in the region of 0.1% to 50% w/w, more preferably 1% to 20% w/w, particularly 1% to 10% w/w, although concentrations both above and below these ranges are envisaged by the present invention.

The formulations of the present invention may further comprise other drugs and/or penetration enhancers. Suitable examples of various drugs are provided above. Further penetration enhancers include lactic acid, DMSO, salicylic acid and oleic acid, of which lactic acid, salicylic acid and oleic acid are individually preferred.

The concentration of enhancer may be any that is effective to permit greater penetration of the nail plate than a similar formulation containing no enhancer. In general, the amount of enhancer will vary between about 0.1% w/w and about 25% W/W, with amounts of between about 1% and 20% and, more preferably, 3% and 15% w/w, often providing good results.

The present invention also provides a method for the treatment of an ungual infection of a nail in a patient in need thereof, comprising applying a preparation of a reducing agent to said nail, followed by applying a preparation of an oxidising agent thereto, said ungual condition being treatable by a drug, said drug being disposed one of said preparations or in a third preparation.

It will be appreciated that the above method preferably comprises any features as specified above in relation to the use, and/or as defined in any of accompanying claims 2 to 43.

The present invention further provides a kit comprising preparations as defined above for the treatment of an ungual infection, and preferably as defined in any of claims 1 to 43.

The present invention will now be further illustrated by the following, non-limiting Examples.

### EXAMPLES

### Examples 1-4

### Materials and Methods

In Examples 1-4, two models were developed to test nail permeability. Model one followed a regimen similar to that of WO 99/49895, where the nail was treated with a penetration enhancer dissolved in a solvent and the % nail weight increase was determined. The penetration enhancers are thought to be keratinolytic and, thus, break the sulphur bonds in the nail, thereby causing it to soften and take up more liquid.

The second model actually measures nail permeability. In this novel model, a radioactive compound, ¹⁴C-mannitol, was used to follow the progress of an agent through the nail after the application of the penetration enhancers. A novel diffusion cell was used to measure mannitol diffusion.

Examples 1-4 use the instruments, materials and methods detailed below.

Materials:

TABLE 1
Suppliers, grade and lot details of materials used in the study.

| Product | Supplier | Address | Grade |
|---|---|---|---|
| Ammonium | Fluka | Sigma-Aldrich | ~60% in water |
| thioglycolate | | Company Ltd, | |
| | | Dorset, UK | |
| Calcium | Fluka | Sigma-Aldrich | ≧98% |
| thioglycolate | | Company Ltd, | |
| | | Dorset, UK | |
| EDTA | Sigma- | Sigma-Aldrich | 99% |
| | Aldrich | Company Ltd, | |
| | | Dorset, UK | |
| Ethanol | BDH | BDH Laboratory | 99.7-100% |
| | | Supplies, Poole, | (Anala ® Grade) |
| | | Dorset, UK | |
| 1,4-Dithio-DL- | Fluka | Sigma-Aldrich | molecular biology |
| threitol | | Company Ltd, | grade ≧99.5% |
| | | Dorset, UK | |
| Glycolic acid | Sigma- | Sigma-Aldrich | ReagentPlus ™ |
| | Aldrich | Company Ltd, | 99% |
| | | Dorset, UK | |
| Hydrogen | Sigma- | Sigma-Aldrich | 35 wt. % in water |
| peroxide | Aldrich | Company Ltd, | |
| solution | | Dorset, UK | |
| L-Cysteine | Sigma- | Sigma-Aldrich | 99% |
| hydrochloride | Aldrich | Company Ltd, | |
| hydrate | | Dorset, UK | |
| Sodium | Sigma- | Sigma-Aldrich | 96.5-103.5% by |
| thioglycolate | Aldrich | Company Ltd, | iodine titration |
| | | Dorset, UK | |
| Thioglycolic | Sigma- | Sigma-Aldrich | ≧98% |
| acid | Aldrich | Company Ltd, | |
| | | Dorset, UK | |
| Urea hydrogen | Sigma- | Sigma-Aldrich | 98% |
| peroxide | Aldrich | Company Ltd, | |
| addition | | Dorset, UK | |
| compound | | | |
| Water | Millipore | Millipore (U.K.) | Milli-Q Gradient |
| | | Limited, | system (18.2 MΩ- |
| | | Gloucestershire | cm resistivity) |
| ³H water | Sigma- | Sigma-Aldrich | ³H water MB11 |
| MB11 stock | Aldrich | Company Ltd, | stock 37 MBq/ml |
| 37 MBq/ml | | Dorset, UK | |
| ¹⁴C mannitol | GEC | GEC Amersham | ¹⁴C mannitol CFA |
| CFA 238 stock | Amersham | Pharmacia. | 238 stock 9.25 |
| 9.25 MBq in | Pharmacia. | St. Giles | MBq in 1.25 ml |
| 1.25 ml | | Bucks | |

### Penetration Enhancers:

As each of the penetration enhancers demonstrated different solubilities, either water, or a mixture of ethanol and water, were used as solvents, in accordance with Table 2, below.

TABLE 2
Solvents used for the penetration enhancers.

| Agent | Solvent |
|---|---|
| Ammonium thioglycolate | 20% ethanol |
| Calcium thioglycolate | Water |
| Sodium thioglycolate | 20% ethanol |
| Thioglycolic acid (TA) | 20% ethanol |
| Glycolic acid | 20% ethanol |
| Dithiothreitol (DTT) | 20% ethanol |
| Cysteine | 20% ethanol |
| Urea-H₂O₂ | Water |

### Methods:

### Nail Swelling Studies:

### Washing and Preparation of Nail Clippings:

Nail clippings of approximately 2 mm in length were obtained from healthy human volunteers (with consent) using nail clippers. These were washed using 70% ethanol (v/v) by vortexing in a 28 ml glass vial using a WhirliMixer™ (Fison's, UK) at maximum speed for 1 min. Clippings were then rinsed in water by vortexing thoroughly again for 1 min. This procedure was repeated three times. Nails were then placed into an open Petri dish and left to dry in a temperature controlled oven at 30 ±2° C. for 24 h. Following oven drying, the nails were weighed and placed into individual wells of a 24-well microbiology plate (Costar®, UK). In all experiments 10 sets of nail clippings were used.

### Application of Penetration Enhancers to Nail Clippings:

The washed nail clippings in the microbiology plate wells were immersed in 1 ml of the penetration enhancer solution for approximately 20 h. Excess solution was removed from all the nail clippings by gently patting them dry using tissue towels. The nail clippings were weighed and weights recorded. If a second penetration enhancer was used, the same procedure was repeated with the second compound and a second weight recorded. In order to estimate the effect of the solvent alone a set of control nails were tested using an identical methodology, but simply immersed in the solvent without the addition of the penetration enhancer.

### Mannitol Nail Penetration Studies:

### Preparation of Penetration Enhancers

The single penetration enhancers were made up in the appropriate solvent and spiked with ¹⁴C mannitol (10 μl of the stock ¹⁴C mannitol). When more than one penetration enhancer was applied to the nails, only the last penetration enhancer was spiked with ¹⁴C mannitol prior to nail application. ¹⁴C mannitol with no penetration enhancer was used as a control.

### Preparation of Nail Diffusion Cell:

Pre-calibrated nail diffusion cells were assembled with full thickness human nail. The receiver fluid, which consisted of a 20% ethanol/water mixture, was put into the receiver well up to the etched mark on the side arm. A magnetic follower was inserted and the cells were then placed on a magnetic stirrer in a water bath maintained at 32° C. Cells were checked for leakage and air bubbles. After a 30 min equilibration period a 1 ml sample was taken from the sampling side arm and placed into a scintillation vial followed by 4 ml of scintillation cocktail and tested on the scintillation counter using the dual mode set up (³H/¹⁴C). This was to ensure that no cells contained any residual radioactivity and these readings were also taken as the background for each cell. The cells were then topped up to the etched mark with fresh receiver fluid, pre-equilibrated at 32° C.

### Normalisation of the Nail Using ³H Water:

After taking the background reading (described in the section above) 50 μl of ³H water (ca. 18000 dpm) was placed on top of the nail in the assembled nail diffusion cell, using a pre-calibrated Gilson pipette. The cells were then occluded immediately with parafilm. After 20 h, a 1 ml sample of the receiver fluid was removed from the side arm using a 1 ml syringe and placed into a scintillation vial followed by 4 ml of scintillation cocktail and tested on the scintillation counter using the dual mode set up (³H/¹⁴C). This value was used to normalise each nail and, if values of radioactivity were considered too high, then cells were rejected due to leaks. A Dixon's Q-test was used to determine if any of the cells were leaking. The ³H water was then dried off using a paper towel and the cell was topped up to the etched mark with fresh receiver fluid, pre-equilibrated at 32° C.

When testing a single penetration enhancer an aliquot of 50 μl of each penetration enhancer (pre-spiked with ¹⁴C mannitol as described above) was pipetted using a pre-calibrated Gilson onto the surface of full thickness human nail. Each cell was then occluded with parafilm. The sampling time points were taken at ca. 20 h, 44 h, 68 h, 92 h, and 116 h. After each time point, a 1 ml sample was taken from the side arm and placed into a scintillation vial followed by 4 ml of scintillation cocktail and tested on the scintillation counter using the dual mode set up (³H/¹⁴C). The cell was then topped up to the etched mark with fresh receiver fluid, pre-equilibrated at 32° C. Each penetration enhancer (and controls) was tested a total of n=5-6 times using the procedure outlined above.

When using more than one penetration enhancer, 50 μl of the penetration enhancer without radioactive mannitol was pipetted using a pre-calibrated Gilson onto the surface of full thickness human nail, each cell was then occluded with parafilm. After 20 h the pre-penetration enhancer was dried off with paper towels and the surface of the nail was washed with de-ionised water (3×1 ml). The second penetration enhancer, pre-spiked with ¹⁴C mannitol, was then applied and the protocol applied to the single penetration enhancer described above followed.

### Example 1

### The Use of Single Nail Penetration Enhancers

### Nail Swelling Studies:

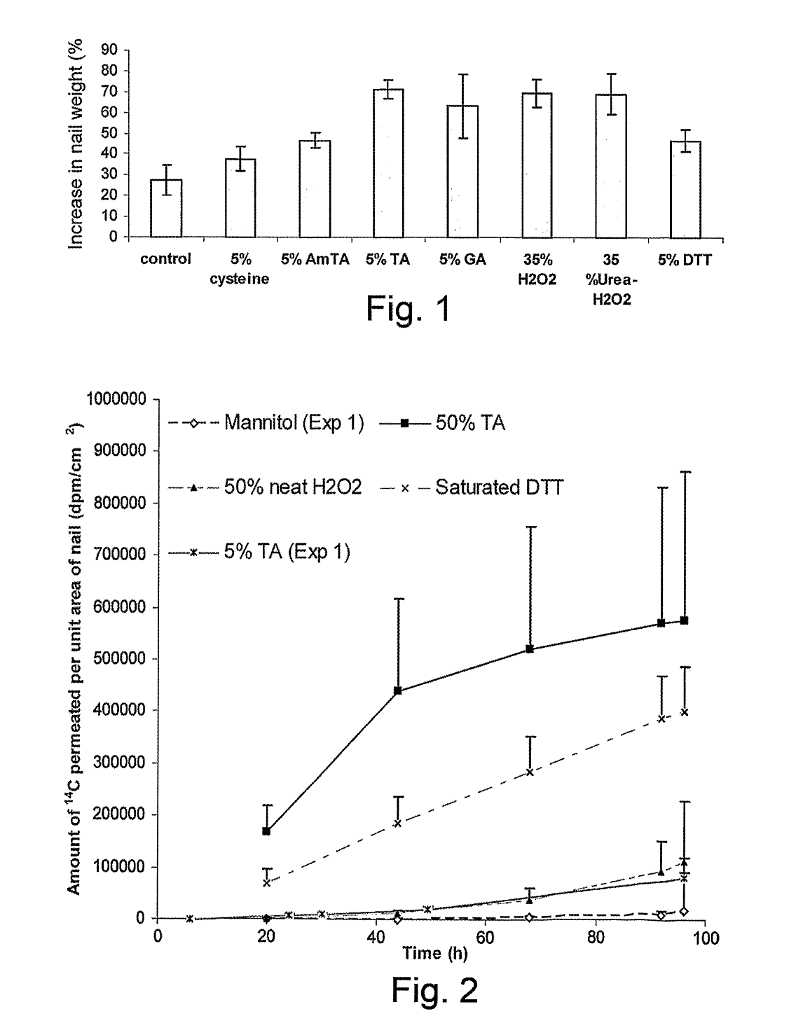

FIG. 1 shows the change in nail weight after 20 h when water (control), cysteine, ammonium thioglycolate (AmTA), thioglycolic acid (TA), glycolic acid (GA), hydrogen peroxide (H₂O₂), urea-hydrogen peroxide (Urea-H₂O₂) and 1,4-dithio-DL-threitol (DTT) were added to nail clippings (mean±standard deviation, n=10). The values are expressed as a percentage of the original nail weight. Although both water and 20% ethanol in water were used as control solutions, there was no significant difference (p>0.05, ANOVA) between the two. Thus, only the water control is shown in FIG. 1.

Thioglycolic acid (TA), glycolic acid (GA), hydrogen peroxide (H₂O₂), urea-hydrogen peroxide (urea-H₂O₂), dithiothreitol (DTT) and ammonium thioglycolate (AmTA) all significantly increased (p<0.05, ANOVA) the weight of the nail clippings after 20 h compared to the control. The increase in nail weight caused by six of the seven penetration enhancers tested infers that the nails are taking up more of the applied fluid. Also noted was the physical change in appearance of treated nails. TA, GA, H₂O₂ and AmTA treated nails were softer than the controls and also lighter in colour.

Cysteine did not significantly increase (p>0.05, ANOVA) nail weight. Thus, the permeation of the fluid into the nails treated with cysteine was not enhanced.

### Mannitol Nail Penetration Studies:

FIG. 2 shows the permeation of ¹⁴C mannitol through full thickness human nail, using different single penetration enhancers (n=6±SD, DTT n=5±SD). Saturated TA (50%) was the best penetration enhancer of mannitol in these experiments. However, when the TA is reduced to a concentration of 5% its penetration enhancing effects drop below that of the DTT. H₂O₂ is not significantly different to the 5% TA (p<0.05, ANOVA) in terms of mannitol diffusion after 96 h.

### Example 2

### The Use of Dual Nail Penetration Enhancers

### Nail Swelling Studies:

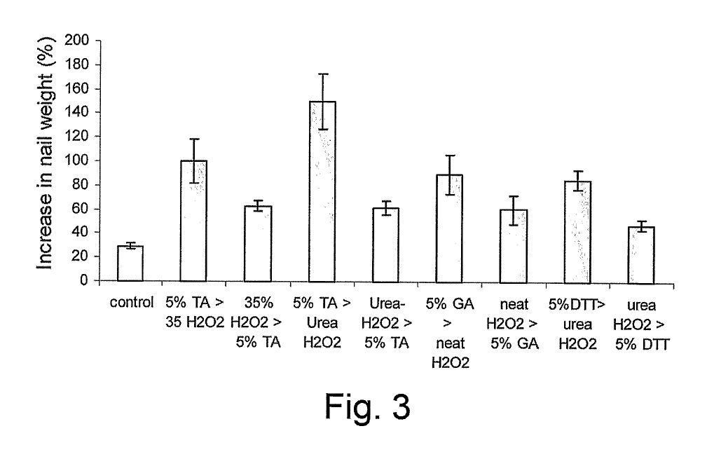

The maximum increase in nail weight with the application of a single penetration enhancer was 71% for 5% TA (shown in FIG. 1). However, the addition of two penetration enhancers, one after another, led to an increase in nail weight of up to 150%, as shown in FIG. 3. FIG. 3 shows the percentage weight gain of nails when treated with 2 penetration enhancers in 2 separate stages (> indicates followed by) in both orders of application (mean±standard deviation, n=10).

Addition of TA and then H₂O₂ or urea-H₂O₂ increased the nail weight significantly (p<0.05, ANOVA) compared to TA alone. However, when H₂O₂ or urea-H₂O₂ was applied before TA it did not significantly increase the nail weight (p>0.05, ANOVA) compared to TA alone. In each of the dual penetration enhancers experiments detailed in FIG. 3 a similar trend was observed. The addition of the reducing agent (e.g. TA, GA, DTT) prior to the oxidising agent (e.g. H₂O₂ or urea-H₂O₂) significantly enhanced the weight increase of the nails compared to the application of the agents in the reverse order i.e. oxidising and then reducing agents. The application of TA followed by urea-H₂O₂ gave the largest increase in weight, whereas the lowest was urea-H₂O₂ followed by DTT.

### Example 3

### The Effect of Penetration Enhancer Concentration

### Nail Swelling Studies:

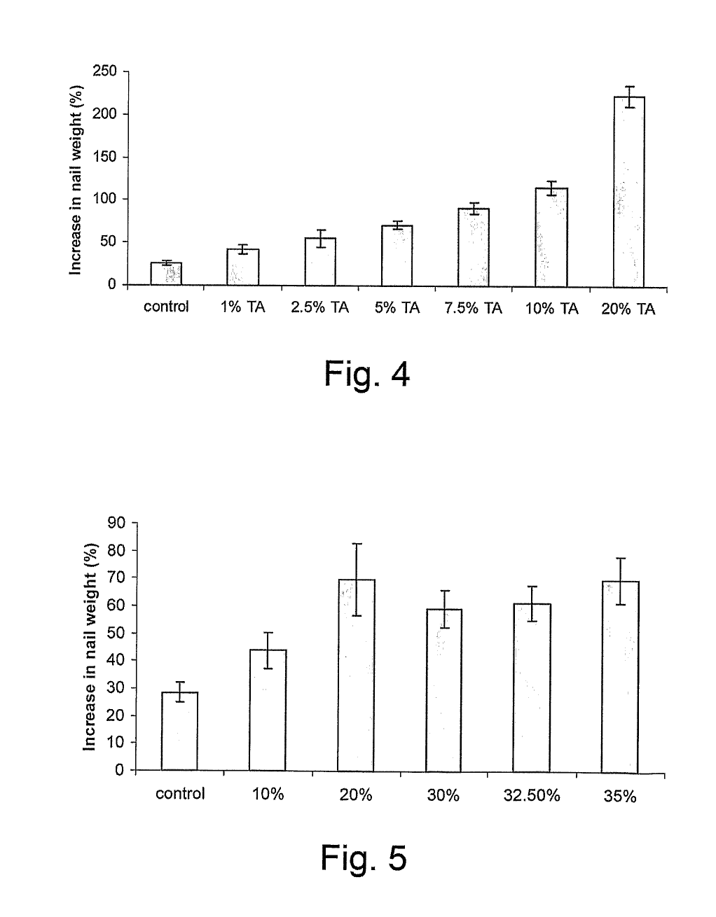

FIG. 4 shows the percentage weight gain of nails after a 20 h period in varying concentrations of TA (mean±standard deviation, n=10). In the case of TA treated nails, increasing concentration of the penetration enhancer led to increased weight gain. The most significant weight gain was seen with nails treated with 20% TA.

FIG. 5 shows the percentage weight gain of nails after a 20 h period in varying concentrations of Urea-H₂O₂. The percentages shown on the x-axis are the total H₂O₂ concentration present (mean±standard deviation, n=10). The concentration of urea-H₂0₂ applied to the nails also influenced weight gain and, thus, permeability of the nail. The application of 20% urea-H₂O₂ was significantly better than 10% in terms of nail weight gain. However, nails treated with increasing concentrations between 20% and 35% H₂O₂ displayed no significant difference (p>0.05, ANOVA) in terms of weight increase.

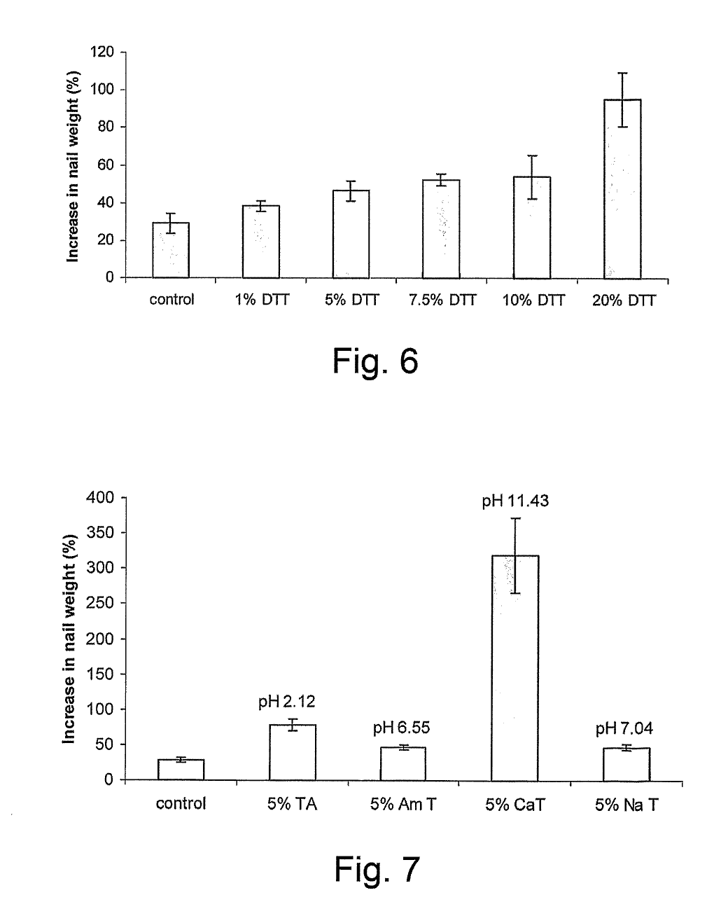

FIG. 6 shows the percentage weight gain of nails after a 20 h period in varying concentrations of DTT (mean±standard deviation, n=10). Nails treated with 1% DTT did show a significant weight increase (p>0.05, ANOVA) compared to nails treated with 5% DTT but, further concentration increases between 5-10% had no significant effect (p<0.05, ANOVA). The greatest effect on nail weight is observed with a DTT concentration of 20%, which was almost double the effect seen with a concentration of 10%.

### Example 4

### The Effect of pH on the Penetration Enhancers

### Nail Swelling Studies:

FIG. 7 shows the percentage weight gain of nails after a 20-hour period in various salts of TA (all at 5% concentration). The numbers above the bars indicate the pH of each solution prior to application (mean±standard deviation, n=10). Regardless of the type of thioglycolate salt employed as the penetration enhancer, the increase in nail weight was significantly larger (p>0.05, ANOVA) than the control. Calcium thioglycolate caused the largest increase in nail weight and, hence, facilitated the greatest amount of liquid to penetrate the nail. The type of salt had a direct influence on the pH of the liquid applied to the nail. TA was applied as a solution with a pH of 2.12 whereas calcium thioglycolate had a pH of 11.43. Thus, whilst all the compounds were effective in enhancing liquid uptake by the nail, a pH>7.1 appeared to be favourable in terms of nail weight increase.

### Examples 5-10

### Materials and Methods

Examples 5-10 use the materials and methods detailed below.

### Redox Measurements:

An ion analysis redox meter (Metrohm, UK) was used to measure the reduction potentials of 0.5 M aqueous preparations of the compounds in deionised water against a stable reference electrode of known potential (Ag/AgCl) at r.t.p.

### Terbinafine Diffusion Studies:

Human nail samples (full thickness) of ca. 3 mm diameter were positioned between the two halves of the specifically designed ChubTur™ diffusion cell and clamped together. The receptor compartment was filled with a previously sonicated suitable buffer system to ensure sink conditions and the cells were fixed on a perspex holder mounted on a magnetic stirrer in a water bath maintained at 32° C. The receptor chamber contents were continuously agitated by small PTFE-coated magnetic followers driven by a submersible magnetic stirrer. A known amount of formulation/drug solution was applied to the surface of the nail (infinite dose) and at regular time intervals samples of buffer were taken from the receptor compartment, replaced with fresh receptor medium and assayed for drug content using HPLC.

### TurChub® Microbiological Tests:

A Sabouraud dextrose agar plate was seeded with Trichophyton rubrum by gently removing mycelium and spores using a sterile swab from a slope culture and transferring them onto the surface of the agar. The plate was incubated at 25° C. for 7 days. The white spores from the surface of the plate were washed off with Ringers solution (20 ml). The spore suspension was filtered through a sterile gauze (Smith+Nephew, Propax, 7.5 cm×7.5 cm, 8 ply gauze swab, BP Type 13) to remove mycelium. The viable count of the spore suspension was assessed and the spore count adjusted to approximately 1×10⁷ cfu/ml, by diluting or concentrating the spores accordingly in a final volume of 20 ml.

Initially, distal nail clippings were obtained from volunteers' toe nails, which had been grown to a minimum length of 3 mm. All volunteers were required to not have used nail varnish or polish on their toe nails within 6 months and have not shown any signs of disease to their nails within 6 months. All volunteers were asked to remove the distal nail sections using either scissors or standard nail clippers. No specific procedures were requested e.g. sterile removal or cleaning of the nails. The nail clippings were then placed into an appropriate container e.g. plastic bag, vial, envelope etc. prior to being placed into an 8 ml bijou bottle per donor/donation and labelled with any details supplied. The samples were stored in a freezer until required.

Using scissors, the nail clippings were then cut into pieces, which were a minimum of 3 mm by 3 mm. The number of pieces obtainable for each nail depends entirely on the size of the original sample, so that a small toe nail may only yield 1 or 2 pieces, whereas a larger toe nail may yield between 3 and 5. The nail clippings were immersed into a 70% ethanol in water solution and vortex-mixed for one minute. The ethanol solution was then decanted and replaced with a fresh 70% ethanol solution and vortex-mixed for a further minute. The ethanol solution was then decanted and replaced with Ringer's solution, vortex-mixed for one minute and decanted and replaced with fresh Ringer's. This process of washing with Ringer's was carried out a total of three times, replacing the wash solution at each phase. Once the washing process was complete, the nail clippings were placed in to a sterile petri dish without a lid and air dried under a laminar flow cabinet for 30 minutes at room temperature. Once the nail clippings were dry, they were placed into new 8 ml bijou bottles (separate bottle per donor, per batch). Nails were also measured for thickness using a pair of callipers.

Distal nail clippings (full thickness nail) were then treated in a diffusion cell with the penetration enhancer system for 20 h with thioglycolic acid followed by 20 h with urea H₂O₂.

Initially the TurChub® cells (lower and upper sections) were sterilised in an autoclave at 121° C. for 15 min. The nail gasket was also sterilised in 100% ethanol and air dried under a laminar flow cabinet prior to mounting the nail/membrane sections. The penetration enhancer treated nails and the gasket were then loaded onto the lower half of the TurChub® cell dorsal side up. A pre-determined volume (calibrated for each cell) of molten SDA agar (maintained at 56° C.) was then loaded into the lower section of the cell After the agar had set a fixed volume (50 μl) of the test organism T. rubrum in Ringers solution was applied onto the surface of the agar. The organism suspension was then encouraged to spread evenly over the surface of the agar by gently rocking the cell from side to side. Excess fluid from the organism suspension was removed from the cell using a syringe and needle. Once the organism was applied to the cell, the nail was mounted and the upper funnel section of the cell was added and fixed into position with springs. The cells are designed so that excess fluid (e.g. from condensation) does not cross-contaminate the agar but accumulates at the bottom of the cell; secondly the cells are orientated in such a way that they avoid false positive results from ‘run-off’ down the agar.

Approximately 100 μl of the active or formulation was applied for 7 days against the test organism T. rubrum. The efficacy of the formulations was determined by measuring the zone of inhibition of growth of T. rubrum in the TurChub™ cell.

### Example 5

### Redox Potential Measurement of Ungual Penetration Enhancers

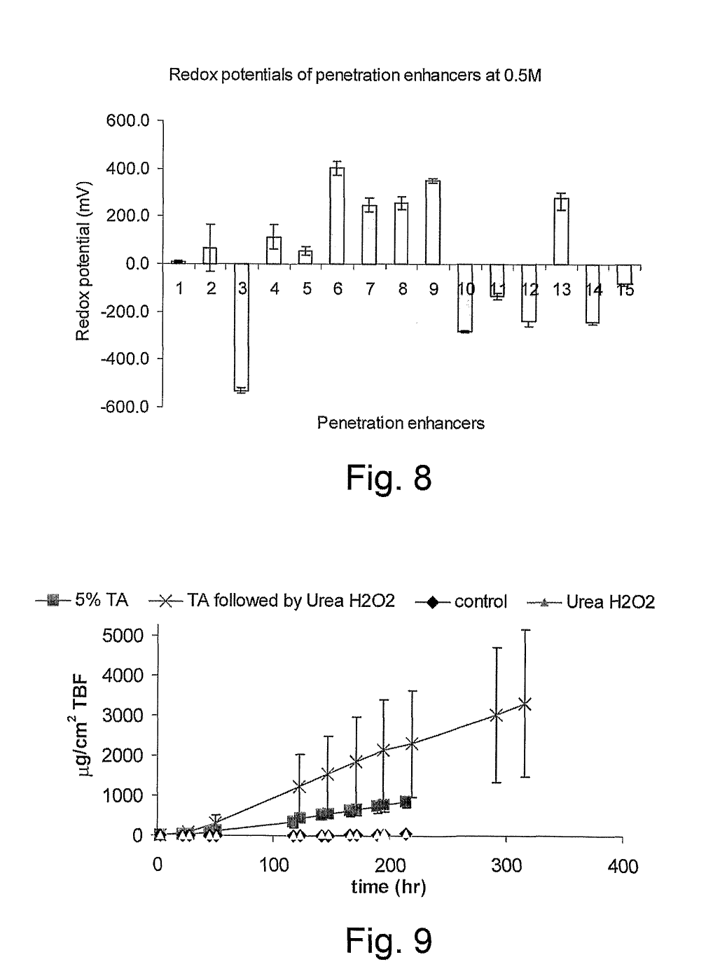

The redox potential of the agents shown to enhance human nail swelling was tested. Reducing agents are expected to have −ve redox potentials whilst oxidising agents will +ve redox potentials (when expressed, in the alternative, as reduction potentials, the sign changes, so that reducing agents will have a +ve potential). The results in Table 3 and in FIG. 8 (±SD, n=3), show that there are clear differences in terms of the redox potentials of the various agents tested.

TABLE 3
Redox potentials and pH values for chemical agents (0.5M
concentration, n = 3):

| | | Mean | | | |
|---|---|---|---|---|---|
| | | Redox | | | |
| | | Potential | Standard | Mean | Standard |
| No. | Chemical | (mV) | deviation | pH | deviation |
| 1 | Resorcinol | 8.6 | 5.3 | 5.67 | 0.42 |
| 2 | Cineol | 67.2 | 100.5 | 5.91 | 0.30 |
| 3 | Calcium thioglycolate | −531.3 | 12.5 | 11.74 | 0.24 |
| 4 | p-coumaric acid | 114.1 | 50.8 | 3.54 | 0.31 |
| 5 | Thiouracil | 53.4 | 18.9 | 6.28 | 0.08 |
| 6 | Potassium persulphate | 402.5 | 28.6 | 2.83 | 0.06 |
| 7 | Glycolic acid | 247.7 | 27.7 | 2.03 | 0.15 |
| 8 | Oxalic acid | 254.8 | 26.0 | 1.33 | 0.08 |
| 9 | Urea hydrogen | 349.7 | 5.3 | 2.72 | 0.31 |
| | Peroxide | | | | |
| 10 | Sodium thioglycolate | −281.7 | 5.3 | 6.90 | 0.05 |
| 11 | Thioglycolic acid | −132.8 | 13.8 | 2.13 | 0.02 |
| 12 | Ammonium | −235.9 | 24.0 | 6.29 | 0.20 |
| | thioglycolate | | | | |
| 13 | Hydrogen peroxide | 277.3 | 48.3 | 6.21 | 0.31 |
| 14 | DTT | −241.0 | 10.9 | 2.64 | 0.53 |
| 15 | Cysteine | −79.3 | 8.2 | — | — |

Those chemical agents that were strongly reducing include the thioglycolate salts, with calcium thioglycolate having the lowest redox potential of all the chemicals (−531.3 mV). The most powerful oxidising agents were those with the highest redox potentials, which include urea-hydrogen peroxide (urea-H₂O₂), hydrogen peroxide (H₂O₂), glycolic acid, oxalic acid and potassium persulphate.

It is evident from this redox data that those combinations of agents that dramatically improved nail swelling (Example 2) were all either strong oxidising or reducing agents. Furthermore, the application order of these agents is important, the greatest nail swelling in Example 2 was observed when the reducing agent was applied to the nail first.

### Example 6

### Effect of Ungual Penetration Enhancing Compound Concentration Upon Redox Potential

The effect of concentration on the redox potential of selected agents was investigated. These included thioglycolic acid, urea-H₂O₂, H₂O₂, and DTT. The results of the titrations are shown in Tables 4-7.

TABLE 4
Mean redox potentials and pH values of TA at concentrations
between 0.001%-20% w/w (n = 3)

| TA | Mean | | | |
|---|---|---|---|---|
| Concentration | Redox | Standard | | Standard |
| (% w/w) | Potential | Deviation | Mean pH | Deviation |
| 20% | −128.7 | 0.1 | 1.61 | 0.04 |
| 10% | −132.8 | 0.6 | 1.83 | 0.14 |
| 7.5% | −132.7 | 0.1 | 1.76 | 0.03 |
| 5% | −131.5 | 0.4 | 1.85 | 0.02 |
| 2.5% | −126.5 | 0.5 | 1.71 | 0.55 |
| 1% | −119.8 | 0.4 | 2.23 | 0.08 |
| 0.1% | −97.4 | 1.4 | 2.66 | 0.13 |
| 0.05% | −85.6 | 0.8 | 2.53 | 0.12 |
| 0.01% | −67.5 | 1.2 | 3.05 | 0.12 |
| 0.005% | −56.6 | 3.8 | 3.12 | 0.19 |
| 0.001% | −47.7 | 5.4 | 2.97 | 0.32 |

Table 4 shows that, at concentrations between 1-20% of TA, there is no concentration effect on the measured redox potential of the solution. At concentrations lower than this (0.001-1%) the redox potential decreases steadily with the drop in concentration. A similar trend was observed when the redox potential of DTT was measured, and only at concentrations below 0.05% did the redox potential of the solution fall (Table 5).

TABLE 5
Mean redox potentials and pH values of DTT
at concentrations between 0.005-12% w/w (n = 2):

| DTT | Mean | | | |
|---|---|---|---|---|
| Concentration | redox | Standard | | Standard |
| (% w/w) | potential | deviation | Mean pH | deviation |
| 10 | −193.1 | 11.45513 | 5.69 | 0.05 |
| 7.5 | −209.65 | 16.33417 | 6.24 | 0.30 |
| 5 | −201.25 | 16.05132 | 6.20 | 0.23 |
| 1 | −185.75 | 10.39447 | 7.01 | 0.06 |
| 0.1 | −189.65 | 0.919239 | 7.59 | 0.09 |
| 0.05 | −169.19 | 0.410122 | 7.80 | 0.12 |
| 0.01 | −134.9 | 4.666905 | 8.00 | 0.02 |
| 0.005 | −122.7 | 3.818377 | 8.07 | 0.45 |

A similar effect is seen with both H₂O₂ and urea-H₂O₂. With the latter at high concentrations of between 5-17.5%, there did not appear to be large differences in redox potential (Table 6). However, as the concentration of urea-H₂O₂ decreased below 5%, the redox potential and thus the oxidising power fell more quickly. The pH of the solution also became more neutral as the concentration of the solution decreased. With H₂O₂ the critical concentration at which the redox potential decreased was 20% (Table 7).

TABLE 6
Mean redox potentials and pH values of Urea-H₂O₂ at
concentrations between 0.0025-17.5 H₂O₂ content w/w (n = 3):

| H2O2 | Mean Redox | | | |
|---|---|---|---|---|
| Content | Potential | Standard | | Standard |
| (% w/w) | (mV | Deviation | Mean pH | Deviation |
| 17.5 | 382.3 | 1.3 | 3.33 | 0.09 |
| 16.25 | 382.9 | 1.5 | 3.87 | 0.08 |
| 15 | 380.3 | 0.8 | 4.04 | 0.03 |
| 10 | 377.9 | 0.5 | 4.23 | 0.12 |
| 5 | 372.3 | 1.4 | 5.10 | 0.11 |
| 0.5 | 293.6 | 11.2 | 5.31 | 0.33 |
| 0.05 | 208.1 | 5.0 | 6.81 | 0.16 |
| 0.025 | 199.5 | 3.4 | 6.56 | 0.04 |
| 0.005 | 191.3 | 5.2 | 6.17 | 1.00 |
| 0.0025 | 189.4 | 6.0 | 6.07 | 0.53 |

TABLE 7
Mean redox potentials and pH values of H₂O₂ at concentrations
between 0.005-35% w/w (n = 3):

| H2O2 | Mean | | | |
|---|---|---|---|---|
| Concentration | redox | Standard | | Standard |
| (% w/w) | potential | deviation | Mean pH | deviation |
| 35 | 444.5 | 0.6 | 2.55 | 0.04 |
| 32.5 | 437.9 | 1.7 | 2.54 | 0.08 |
| 30 | 430.4 | 3.4 | 2.64 | 0.02 |
| 20 | 402.8 | 1.3 | 3.16 | 0.01 |
| 10 | 357.2 | 3.7 | 3.79 | 0.02 |
| 1 | 231.6 | 9.1 | 6.17 | 0.03 |
| 0.1 | 193.5 | 3.8 | 6.71 | 0.06 |
| 0.05 | 200.4 | 7.8 | 6.78 | 0.12 |
| 0.01 | 153.0 | 18.4 | 7.46 | 0.26 |
| 0.005 | 142.3 | 6.6 | 7.56 | 0.08 |

### Example 7

### Effect of Ungual Penetration Enhancing Compound pH Upon Redox Potential

To investigate whether the pH of the solution has an effect on the redox potential of the penetration enhancers, a pH range was investigated for two of the compounds: 5% TA solution and urea-H₂O₂ with a H₂O₂ content of 15%. TA was tested using a pH range of 2-11 (pH was adjusted using NaOH solution). The urea-H₂O₂ solution was tested at pH's between 2-10 (pH was adjusted using HCl and NaOH solutions).

The redox potential of TA almost increased one order of magnitude over the pH range tested (Table 8). In contrast, the redox potential of urea-H₂O₂ complex decreased steadily until pH 10 was reached, where the redox potential was seen to drop sharply (Table 9).

TABLE 8
The mean redox potential of TA solutions at pH values ranging
from pH 2-11 (n = 3).

| Approximate | Mean redox | Standard |
|---|---|---|
| pH of TA | potential | deviation |
| 2 | −70.4 | 20.4 |
| 3 | −133.1 | 13.3 |
| 4 | −166.7 | 24.4 |
| 5 | −179.5 | 33.7 |
| 6 | −192.9 | 37.3 |
| 8 | −272.3 | 49.4 |
| 10 | −386.5 | 52.0 |
| 11 | −433.8 | 50.6 |

TABLE 9
The mean redox potential of Urea-H₂O₂ solutions at pH values ranging
from pH 2-10 (n = 3 + SD).

| Approximate pH of | Redox potential | Standard |
|---|---|---|
| Urea-H₂O₂ | (mV) | deviation |
| 2 | 459.8 | 2.0 |
| 4 | 334.1 | 5.5 |
| 6 | 284.0 | 1.8 |
| 8 | 194.2 | 0.7 |
| 10 | 16.1 | 1.2 |

### Example 8

### Penetration Enhancement of a Model Pharmaceutical Compound

FIG. 9 is a graphical representation of terbinafine diffusion through a human nail after pre-treatment with either 50:50 ethanol/water (control), urea-H₂O₂ or thioglycolic acid (TA) alone, and the two penetration enhancers in combination. The results demonstrate the ability of the dual penetration enhancer system to increase the difflusion of a typical drug.

When the nails were soaked in a 50:50 ethanol/water (control) solution for 20 h and the drug applied, only 44±13 μg per cm² penetrated the nail after 214 h. The application of the oxidising agent, urea-hydrogen peroxide had no significant effect (p>0.05, ANOVA) on the amount of drug penetrating the nail, allowing 47±33 μg per cm² to cross the barrier. Applying thioglycolic acid to the nail prior to the application of terbinafine allowed 861±135 μg per cm² of the drug to penetrate, but this was lower than when thioglycolic acid and urea-H₂O₂ were applied sequentially prior to terbinafine, as this allowed 2308±1332 μg per cm² after 219 h.

### Example 9

### Effect of Application Time on the Effectiveness of the Dual Application Penetration Enhancer System

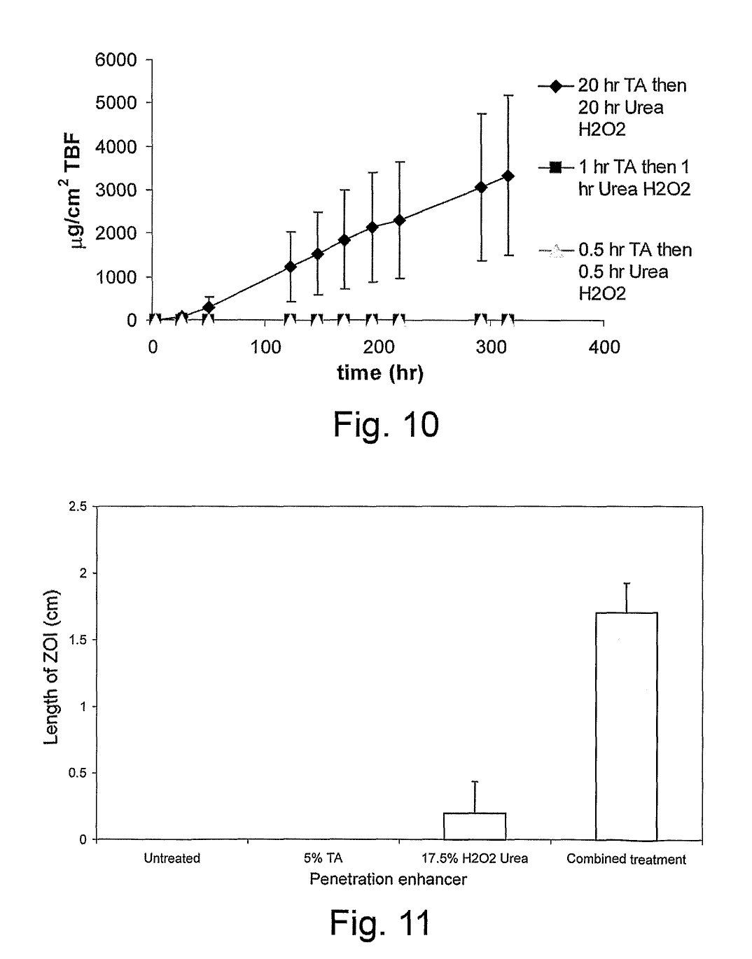

FIG. 10 is a graphical presentation of terbinafine diffusion through a human nail after the pre-treatment with thioglycolic acid (TA) and then urea-H₂O₂ for different periods of time.

Applying the penetration enhancer system for either 30 min or 1 h allowed less than 10 μg per cm² of drug to penetrate the nail in both cases. In contrast, using the dual penetration enhancer system for up to 20 h each increased the amount of terbinafine penetrating the human nail to 3329±1842 μg per cm² after 315 h.

### Example 10

### Use of the Penetration Enhancer System to Improve the Performance of Commercial Antifungal Formulations

The effects of the dual penetration enhancer system on the penetration of amorolfine from the commercial pharmaceutical lacquer Loceryl® was tested using a microbiological assay. The assay assessed the efficacy of the formulation either alone or with the pre-treatment of either thioglycolic acid, urea-hydrogen peroxide or both against the predominant micro-organism found in onychomycotic nails, T. rubrum.

FIG. 11 is a comparison of the average length of the zone of inhibition (ZOI) using the TurChub® test cell system with the organism T. rubrum, after applying pre-treated full thickness distal nail clippings with a variety of penetration enhancing systems, and then applying 100 μl of a standard Loceryl lacquer to the surface of each and incubating for seven days (n=4 ±SD).

FIG. 11 demonstrates that, when amorolfine was either applied alone or after the nail was pre-treated for 20 h with a single reducing agent, thioglycolic acid, the growth of the micro-organism was not prevented i.e. there was no detectable zone of inhibition. When an oxidising agent, urea-hydrogen peroxide was used to pre-treat the nail a small zone of inhibition was detected. However, when both the oxidising and reducing agent were sequentially applied to the nail prior to the antifungal drug, a zone of inhibition of almost 2 cm was observed. These results highlight the application of both an oxidising and reducing agent significantly enhances the microbiological kill attained using a commercially available ungually applied formulation.

1. A method for the treatment of an ungual condition treatable by a drug in a patient in need thereof, wherein a first preparation of a reducing agent and a second preparation of an oxidizing agent are separately disposed and sequentially administered to the nail of said patient in the order of first preparation followed by second preparation, said drug being disposed in said first preparation said second preparation, or, optionally, in a third preparation, said drug being additional to said reducing and said oxidising agents.

2. The method of claim 1, wherein a simple combination of said first and second preparations would lead to an extreme reaction, and wherein each agent is individually pharmaceutically acceptable to apply to a nail.

3. The method of claim 1, wherein said reducing agent is selected from the group consisting of: ammonium thioglycolate, calcium thioglycolate, sodium thioglycolate, thioglycolic acid, and dithiothreitol, ascorbic acid, hydroquinone, mercaptoethanol, glutathione, L-cysteine, taurine, aminomethanesulfonic acid, cysteic acid, cysteinesulfinic acid, ethanedisulfonic acid, ethanesulfonic acid, homotaurine, hypotaurine, isethionic acid, mercaptoethanesulfonic acid, N-methyltaurine, and simple derivatives thereof.

4. The method of claim 3, wherein said derivative is a salt.

5. The method of claim 3, wherein said reducing agent is thioglycolic acid or a derivative thereof.

6. The method of claim 1, wherein said oxidizing agent is selected from the group consisting of: urea, hydrogen peroxide, potassium persulfate, thiouracil, p-coumeric acid, glycolic acid, oxalic acid, cineol, peroxydone, chlorine dioxide, ammonium dichromate, ammonium nitrate, ammonium perchlorate, ammonium permanganate, barium bromate, barium chlorate, barium peroxide, cadmium chlorate, calcium chlorate, calcium chromate, calcium perchlorate, chromium nitrate, cobalt nitrate, silver oxide, periodic acid, and pyridine dichromate.

7. The method of claim 1, wherein said oxidizing agent is hydrogen peroxide.

8. The method of claim 1, wherein said oxidizing agent is an addition compound of hydrogen peroxide and urea.

9. The method of claim 1, wherein said drug is present in one or both of said preparations.

10. The method of claim 1, wherein said drug is present in a third preparation.

11. The method of claim 1, wherein the preparations are liquid.

12. The method of claim 1, wherein said reducing agent is prepared as an alkaline preparation, with a pH of between 7 and 14.

13. The method of claim 12, wherein the pH is between 8 and 13.

14. The method of claim 12, wherein the pH is between 9 and 12.

15. The method of claim 1, wherein the pH of said oxidizing agent is between 1 and 7.

16. The method of claim 15, wherein the pH is between 2 and 5.

17. The method of claim 1, wherein the preparations are aqueous.

18. The method of claim 1, comprising propylene glycol as a bulking agent.

19. The method of claim 1, wherein thioglycolic acid is conjugated with said drug.

20. The method of claim 1, wherein said drug is selected from the group of anti-fungal drugs consisting of: amorolfine, miconazole, ketoconazole, itraconazole, fluconazole, econazole, ciclopirox, oxiconazole, clotrimazole, terbinafine, naftifine, amphotericin, griseofulvin, voriconazole, flucytosine, nystatin and pharmaceutically acceptable salts and esters thereof.

21. The method of claim 1, wherein said drug is terbinafine.

22. The method of claim 1, wherein said drug is amorolfine.

23. The method of claim 1, wherein drug is selected from the group of anti-psoriatic drugs consisting of: corticosteroids, 5-fluorouracil, methotrexate, etretinate, cyclosporin, tacrolimus, and derivatives thereof.

24. The method of claim 1, wherein said oxidizing agent has a reduction potential, and wherein said reduction potential of 0.5 M of the said oxidizing agent in deionised water, when measured against an Ag/AgCl reference electrode, is less than −50 mV.

25. The method of claim 24, wherein said reduction potential is less than −100 mV.

26. The method of claim 24, wherein said reduction potential is less than −200 mV.

27. The method of claim 1, wherein said reducing agent has a reduction potential, and wherein said reduction potential of 0.5 M of said reducing agent in deionised water, when measured against an Ag/AgCl reference electrode, is greater than +20 mV.

28. Use according to The method of claim 27, wherein the said reduction potential is greater than +50 mV.

29. The method of claim 27, wherein said reduction potential is greater than +75 mV.

30. The method of claim 1, wherein said reducing agent is conjugated with said drug.

31. The method of claim 1, wherein said oxidizing agent is conjugated with said drug.

32. The method of claim 1, wherein at least one of said preparations is selected from the group consisting of: creams, ointments, gels, solutions, lotions, foams, mousses, sprays, pastes, dressings, powders, premixes, non-aqueous lacquers and aqueous lacquers.

33. The method of claim 1, wherein said preparations are each individually selected from the group consisting of: creams, ointments, gels, solutions, lotions, foams, mousses, sprays, pastes, dressings, powders, premixes, non-aqueous lacquers and aqueous lacquers.

34. The method of claim 1, wherein at least one of said preparations is a water-based gel.

35. The method of claim 1, wherein all of said preparations are water-based gels.

36. The method of claim 1, wherein at least one of said preparations is a water-based lacquer.

37. The method of claim 1, wherein all of said preparations are water-based lacquers.

38. The method of claim 1, wherein said first preparation is applied at least 10 hours before the oxidising said second preparation.

39. The method of claim 1, wherein said first preparation is applied at least 15 hours before the said second preparation.

40. The method of claim 1, wherein said first preparation is applied at least 20 hours before the said second preparation.

41. The method of claim 1, wherein said third preparation is applied within an hour after application of said oxidizing agent preparation.

42. The method of claim 1, wherein said third preparation is applied within 20 minutes after application of said second preparation.

43. The method of claim 1, wherein said third preparation is applied immediately after application of said second preparation.

44. A method for the treatment of an ungual infection of a nail in a patient in need thereof, comprising applying a preparation of a reducing agent to said nail, followed by applying a preparation of an oxidizing agent thereto, said ungual condition being treatable by a drug, said drug being disposed in one of said preparations or in a third preparation.

45. canceled

46. A kit comprising a first preparation of a reducing agent and a second preparation of an oxidizing agent separately disposed therein, a drug to treat an ungual condition being disposed in said first preparation, said second preparation, or, optionally, in a third preparation, said drug being additional to said reducing and said oxidizing agents.
